(19) United States
(12) Patent Application Publication
(10) Pub. No.: US 2013/0096028 A1
(43) Pub. Date: Apr. 18, 2013

(54) CELL CAPABLE OF EXPRESSING TSLP CONSTANTLY AND AT HIGH LEVEL, AND METHOD FOR SCREENING FOR TSLP MODULATOR UTILIZING THE CELL

(71) Applicants: Noriyasu Hirasawa, Miyagi (JP); Michihiro Hide, Hiroshima (JP)

(72) Inventors: Noriyasu Hirasawa, Miyagi (JP); Michihiro Hide, Hiroshima (JP)

(73) Assignee: TOHOKU UNIVERSITY, Miyagi (JP)

(21) Appl. No.: 13/806,043

(22) PCT Filed: Jun. 13, 2011
(86) PCT No.: PCT/JP2011/003329

§ 371 Date: Dec. 21, 2012

(30) Foreign Application Priority Data
Jun. 28, 2010 (JP) 2010-146262

(51) Int. Cl. C12Q 1/02

(52) U.S. Cl. CPC C12Q 1/025
USPC 506/9; 435/7.92; 435/70.1

(57) ABSTRACT

The present invention relates to a mouse epithelial cell line constantly producing TSLP and use thereof. More specifically, the present invention relates to a method for screening for a TSLP modulator using a mouse epithelial cell line (KCHM-1) constantly producing TSLP, and a method for producing wild-type TSLP using the cell line.

### TECHNICAL FIELD

The present invention relates to a mouse epithelial cell line constantly producing TSLP and use thereof. More specifically, the present invention relates to a method for screening for a TSLP modulator using a mouse epithelial cell line (KCHM-1) constantly producing TSLP, and a method for producing wild-type TSLP using the cell line.

### BACKGROUND ART

Thymic stromal lymphopoietin (TSLP) is an IL-7-like cytokine isolated from the culture supernatant of thymic stromal cells. TSLP has received attention as a master switch for the onset of allergy. When locally expressed in the lung, the skin, or the like, TSLP initiates Th2-type immune response to induce allergic inflammations such as asthma or atopic dermatitis.

According to the previous reports, TSLP production is induced in in-vitro experimental systems from airway epithelial cells, skin keratinocytes, fibroblasts, or mast cells upon stimulation with TNF-α, IL-4, LPS, dsRNA, or the like (Non Patent Literatures 1 to 3).

In vitro study has revealed that the allergy-inducing effect of TSLP is based on, for example, its activation of dendritic cells, which in turn induce differentiation into Th2 cells (Non Patent Literature 4). A complicated mechanism, however, underlies inflammations including allergic diseases. Also, much remains unknown about the mechanism of action of TSLP.

Reportedly, TSLP is highly expressed in keratinocytes in the skin lesions of many atopic dermatitis patients. Patent applications have also been filed for TSLP as a novel preventive or therapeutic target of allergic diseases.

Such patent applications relate to, for example, a method for treating an immune disorder by regulating the activity of dendritic cells using a TSLP agonist or antagonist, or a pharmaceutical composition comprising recombinant TSLP rendered resistant to protease (Patent Literatures 1 and 2).

Analysis on the mechanism of action of TSLP or search for TSLP modulators is considered very useful in providing novel methods for preventing or treating allergic diseases. Although TSLP is easily detected in vivo, in-vitro TSLP production is difficult to induce from keratinocytes. Also, TSLP expression levels are very low in mammalian cell lines known in the art. The recombinant expression system of TSLP has also been constructed, but differs in the mechanism underlying TSLP production from the native one. This system therefore fails to show the accurate control mechanism of TSLP production. In addition, currently commercially available TSLP is recombinant TSLP without sugar chains, which may differ in biochemical properties from TSLP produced by mammalian cells.

For these reasons, there has been a demand for the construction of cell systems highly expressing native TSLP useful in analysis on the mechanism of action of TSLP or search for TSLP-targeting drugs.

### CITATION LIST

### Patent Literature

Patent Literature 1: National Publication of International Patent Application No. 2005-516606
Patent Literature 2: National Publication of International Patent Application No. 2005-505293

### Non Patent Literature

Non Patent Literature 1: Bogiatzi S.I. et al., J. Immunol. (2007) 178: 3373-3377
Non Patent Literature 2: Kato A. et al., J. Immunol. (2007) 179: 1080-1087
Non Patent Literature 3: Allakhverdi Z et al., J. Exp. Med. (2007) 204: 253-258
Non Patent Literature 4: Soumelis V., Nat. Immunol. (2002) 3: 673-680

### SUMMARY OF INVENTION

### Technical Problem

An object of the present invention is to provide a novel tool for developing TSLP-targeting anti-inflammatory agents or antiallergic agents based on the control mechanism of inflammation or allergy mediated by TSLP.

### Solution to Problem

The present inventors have found that the mouse skin epithelial cell line KCMH-1 established by Professor Hide (Faculty of Medicine, Hiroshima University) produces TSLP in large amounts even without stimulation. Although TSLP is easily detected in vivo, in-vitro TSLP production is difficult to induce from keratinocytes. Thus, TSLP production systems using mammalian epithelial cell lines known in the art have not yet been established.

TSLP is known to be closely related to the onset of allergy or inflammation. The cell line is very useful in elucidating the TSLP-mediated pathogenesis of this allergy or inflammation or establishing an in-vitro evaluation system for TSLP-targeting drugs.

Specifically, the present invention relates to a method for screening for a TSLP modulator, comprising: allowing a test substance to act on a KCMH-1 cell line specified by accession No. FERM BP-11368 or variant thereof having a substantially equivalent TSLP productivity thereto; and measuring the obtained amount of TSLP produced.

The screening method of the present invention comprises, for example, the following steps:
1) culturing the KCMH-1 cell line or variant thereof having a substantially equivalent TSLP productivity thereto in the presence and in the absence of the test substance; and
2) comparing the amount of TSLP produced between in the presence and in the absence of the test substance.

In the method, the amount of TSLP produced can be measured using an anti-TSLP antibody specifically binding to TSLP. For example, an immunological method such as Western blotting, dot blotting, slot blotting, ELISA, RIA, or flow bead array assay can be used.

The amount of TSLP produced may be evaluated at the gene (mRNA) level. Such a method is also encompassed in the phrase “measuring the amount of TSLP produced” described above. The gene expression level can be measured using a method known in the art, such as nucleic acid hybridization using samples immobilized on solid phases such as gene chips or arrays, RT-PCR, real-time PCR, subtraction method, differential display method, differential hybridization, and cross-hybridization.

In one embodiment, the TSLP modulator is a TSLP production inhibitor. In the case of screening for the TSLP production inhibitor, if the amount of TSLP produced in the presence of a test substance is significantly lower than that in the absence of the test substance, the test substance is selected as a TSLP production inhibitor candidate.

Examples of the TSLP production inhibitor can include antiallergic agents and anti-inflammatory agents.

The present invention also provides a kit for screening for a TSLP modulator, comprising a KCMH-1 cell line or variant thereof having a substantially equivalent TSLP productivity thereto.

The kit may further comprise an anti-TSLP antibody or an anti-TSLP antibody and a secondary antibody capable of specifically binding to the anti-TSLP antibody.

The present invention further provides a method for producing TSLP, comprising culturing a KCMH-1 cell line specified by accession No. FERM BP-11368 or variant thereof having a substantially equivalent TSLP productivity thereto.

### Advantageous Effects of Invention

According to the present invention, wild-type TSLP can be obtained conveniently.

According to the present invention, the TSLP-modulating activity of a test substance can be evaluated conveniently using a cultured cell system. A substance having TSLP-modulating activity may be used as an antiallergic agent or an anti-inflammatory agent. Thus, the present invention can be used in a convenient and inexpensive screening system for such drugs. Particularly, the KCMH-1 cell line used in the present invention is derived from a keratinocyte and as such, is useful in screening for therapeutic or preventive drugs for cutaneous allergy or searching for the mechanism underlying this allergy.

### BRIEF DESCRIPTION OF DRAWINGS

FIG. 1 shows results of comparing the amount of TSLP produced among KCMH-1 cells, PAM212 cells, and HaCaT cells (in the diagram, the results of KCMH-1 cells, PAM212 cells, and HaCaT cells are shown in this order from the left).

FIG. 2 shows the amount of TSLP produced in KCMH-1 cells after stimulation with dexamethasone or FK506 (in the diagram, the results of cells supplemented with no stimulator (control), 0.1 μM dexamethasone, 1.0 μM dexamethasone, 0.1 μM FK506, or 1.0 μM FK506 are shown in this order from the left).

FIG. 3 shows the effect of an RXR agonist HX-600 on the TSLP production of KCMH-1 cells (right: supplemented with 1 μM HX-600, left: not supplemented).

FIG. 4 shows results of comparing the effects of various tyrosine kinase inhibitors on the TSLP production of KCMH-1 cells (in the diagram, the results of DMSO (control), 3 μM herbimycin A, 3 μM PP2, 100 μM piceatannol, 100 μM AG490, and 30 μM WHI-P154 are shown in this order from the left).

FIG. 5 shows results of comparing the effects of various serine-threonine kinase inhibitors on the TSLP production of KCMH-1 cells (in the diagram, the results of DMSO, 1 μM U0126, 10 μM SB203580, 30 μM SP600125, 100 nM wortmannin, 10 μM BAY11-7082, and 3 μM Go-6976 are shown in this order from the left).

The present specification encompasses the contents described in the specification of Japanese Patent Application No. 2010-146262, which serves as a basis for the priority of the present application.

### Description of Embodiments

### 1. KCMH-1 Cell Line

The “KCMH-1 cell line” used in the present invention is a keratinocyte cell line established from mouse skin cancer cells by Professor Hide (Faculty of Medicine, Hiroshima University), as described later in Reference Examples. This cell line was domestically deposited on May 21, 2010 under accession No. FERM P-21965 with International Patent Organism Depositary (IPOD), the National Institute of Advanced Industrial Science and Technology (AIST) (Tsukuba Central 6, 1-1-1 Higashi. Tsukuba, Ibaraki, Japan) and then transferred and internationally deposited therewith on Apr. 25, 2011 under accession No. FERM BP-11368.

The present inventors have found that this KCMH-1 cell line constantly produces TSLP and that the amount of TSLP produced is much higher than that induced by the stimulation of other keratinocytes.

Although TSLP, as described above, is easily detected in vivo, in-vitro TSLP production is difficult to induce from keratinocytes. Thus, TSLP production systems using mammalian epithelial cell lines known in the art have been unknown so far. Cells may be forced to highly express TSLP by TSLP gene transfer. The resulting cells transgenic for the TSLP gene, however, differ in the control mechanism of TSLP production from intact cells and therefore, do not accurately reflect the in-vivo TSLP-mediated pathogenesis of allergy or inflammation.

The KCMH-1 cell line of the present invention is free from these problems and as such, is very useful in cultured cell systems for elucidating the in-vivo TSLP production mechanism or searching for TSLP modulators.

In the present invention, in addition to the KCMH-1 cell line or progeny thereof, a variant thereof (hereinafter, also referred to as a “KCMH-1 cell variant”) may be used as long as the variant has a substantially equivalent TSLP productivity thereto. Examples of the “KCMH-1 cell variant” can include auxotrophic variants and drug-resistant variants thereof, and transgenic strains.

### 2. TSLP

Thymic stromal lymphopoietin (TSLP) is a 121-amino acid IL-7-like cytokine isolated from the culture supernatant of thymic stromal cells. TSLP has received attention as a master switch for the onset of allergy. When locally expressed in the lung, the skin, or the like, TSLP initiates Th2-type immune response to induce allergic inflammations such as asthma or atopic dermatitis. In vitro study has revealed that the allergy-inducing effect of TSLP is based on, for example, its activation of dendritic cells, which in turn induce differentiation into Th2 cells.

According to the previous reports, TSLP production is induced in in-vitro experimental systems from airway epithelial cells, skin keratinocytes, fibroblasts, or mast cells upon stimulation with TNF-α, IL-4, LPS, dsRNA, or the like. By contrast, TSLP production is reportedly inhibited by dexamethasone, but not by cyclosporine A or FK506.

For example, Kato et al. added IL-4 (100 ng/ml), IL-13 (100 ng/ml), interferon-y (100 ng/ml), interferon-β (1000 U/ml), toll-like receptor 3 ligand dsRNA (25 μg/ml), and TNF-α (100 ng/ml) to 3×10⁴ normal human airway epithelial cells cultured for 48 hours, and analyzed the expression level of TSLP mRNA and the amount of TSLP in each culture solution over time. They reported that TSLP was induced by dsRNA, TNF-α+IL-4, or TNF-α+IL-13 stimulation (Kato, A. et al., J. Immunol. (2007) 179: 1080-1087).

Allakhverdi et al. stimulated primary human airway epithelial cells with IL-1 (10 ng/ml), TNF-α (25 ng/ml), PGN (100 μg/ml), poly I:C (50 μg/ml), LPS (1 μg/ml), or CpG (5 μM) for 48 hours and assayed the amount of TSLP in the supernatant of the culture solution and the expression of toll-like receptor mRNA in each cell. They showed that the primary human airway epithelial cells principally expressed TLR2 and TLR3 and that their respective ligands PGN and poly I:C increased the production of TSLP. Furthermore, TSLP receptor-expressing cells in bronchoalveolar lavage fluids were cultured for 3 days in the presence of the culture supernatant (SNT) of stimulated primary human airway epithelial cells and in the presence or absence of TSLP-neutralizing antibodies, and cell growth thereof was analyzed. Similarly, IL-13 in the supernatant of the culture solution was assayed 24 hours after stimulation. As a result, TSLP was shown to have cell growth activity and IL-13 production-inducing activity. Thus, TSLP is likely to also participate in the aggravation of allergy or inflammation caused by infection in the respiratory system and as such, can serve as a target molecule of respiratory diseases accompanied by infection (Allakhverdi, Z. et al., J. Exp. Med. (2007) 204: 253-258).

Meanwhile, Le et al. cultured for 24 hours normal human keratinocytes stimulated with poly I:C in the presence of IL-4 (100 ng/ml), IL-13 (100 ng/ml), or TNF-α (20 ng/ml), and normal human keratinocytes supplemented with dexamethasone, cyclosporine A, FK506, and its solvent DMSO simultaneously with such stimulation, and then assayed TSLP in the supernatant of each culture solution. They reported that TSLP production was inhibited by dexamethasone, but not by cyclosporine A or FK506 (Allergy 2009, 64, 1231-1232).

Also, the high expression of TSLP has been reported in patients with allergy-related diseases such as atopic dermatitis or rheumatism.

For example, Ozawa et al. reported that TSLP was produced in larger amounts in synovial fibroblasts derived from rheumatism patients (RA) and osteoarthropathy patients (OA) (Mod. Rheumatol 2007, 17, 459-463). They stimulated synovial fibroblasts (2.5×10⁷ cells/well) obtained from rheumatism patients (RA) and osteoarthropathy patients (OA) with LPS (1 μg/ml) or poly I:C (10 μg/ml) in the presence of IMD-0354 (IMD, IκB kinase inhibitor, 10 μM) or dexamethasone (0.1 nM), and measured the amount of TSLP in each culture supernatant after 24-hour culture. Also, change in IκBα level caused by LPS or poly I:C stimulation was analyzed over time. From these results, they reported that TLR ligands induced TSLP production and that TSLP could serve as a target molecule not only for allergic diseases but articular rheumatism.

These findings suggest that TSLP is very useful as a therapeutic target for inflammation or allergy including respiratory diseases, atopic dermatitis, and rheumatism. This indicates that the KCMH-1 cell line of the present invention that can be used in cultured cell systems for evaluating the regulation or control of TSLP production works as a useful in-vitro screening system for TSLP modulators including therapeutic drugs for inflammation or allergy.

### 3. Method for Screening for TSLP Modulator

### 3.1 TSLP Modulator

On the basis of the findings described above, the present invention provides a method for screening for a TSLP modulator using the KCMH-1 cell line.

In this context, the “TSLP modulator” means a substance (compound, composition, etc.) capable of directly or indirectly regulating or controlling the amount of TSLP produced, thereby regulating, preventing, or treating the symptoms of inflammation, allergy, or the like, mediated by TSLP. In other words, the “TSLP modulator” is a TSLP activity-controlling agent or a TSLP-targeting drug. The TSLP modulator encompasses both of those reducing and promoting the amount of TSLP produced. Since TSLP is generally a mediator of inflammation or allergy, the “TSLP production inhibitor”, which reduces the amount of TSLP produced, is useful as an antiallergic agent or an anti-inflammatory agent. As described later in Examples, the present inventors have confirmed that dexamethasone remarkably reduces the amount of TSLP produced in the KCMH-1 cell line.

In the present invention, in addition to the KCMH-1 cell line, a variant thereof having a substantially equivalent TSLP productivity thereto may be used. Specifically, a test substance is allowed to act on the KCMH-1 cell line or variant thereof having a substantially equivalent TSLP productivity thereto; and the obtained amount of TSLP produced is measured, thereby evaluating whether the test substance can be used as a TSLP modulator.

### 3.2 Culture of KCMH-1 Cell

The KCMH-1 cell or variant thereof having a substantially equivalent TSLP productivity thereto is adhesively cultured in a basal medium usually used in animal cell culture. Examples of the basal medium that can be used include a DMEM medium, a BME medium, a BGJb medium, a CMRL 1066 medium, a Glasgow MEM medium, an Improved MEM Zinc Option medium, an IMDM medium, a Medium 199 medium, an Eagle MEM medium, an MEMu medium, a Dulbecco MEM medium, a Ham's medium, an RPMI 1640 medium, a Fischer's medium, a McCoy's medium, a William's E medium, and mixed media thereof.

The basal medium may be supplemented with various nutrient sources necessary for cell maintenance and growth and each component necessary for the induction of cell differentiation.

Examples of the nutrient sources can include: carbon sources such as glycerol, glucose, fructose, sucrose, lactose, honey, starch, and dextrin; hydrocarbons such as fatty acid, fat and oil, lecithin, and alcohol; nitrogen sources such as ammonium sulfate, ammonium nitrate, ammonium chloride, urea, and sodium nitrate; inorganic salts such as common salt, potassium salt, phosphate, magnesium salt, calcium salt, iron salt, and manganese salt; monopotassium phosphate, dipotassium phosphate, magnesium sulfate, sodium chloride, ferrous sulfate, sodium molybdate, sodium tungstate, and manganese sulfate; various vitamins; and amino acids.

Examples of other components that can be added, if necessary, to the medium can include pyruvic acid, pyruvic acid, amino acid-reducing agents such as β-mercaptoethanol, serum, and serum substitutes. Examples of the serum substitutes include albumin (e.g., lipid-rich albumin), transferrin, fatty acid, insulin, collagen precursors, trace elements, β-mercaptoethanol, 3′-thiol glycerol, and commercially available products such as Knockout Serum Replacement (KSR), Chemically-defined Lipid concentrated (Gibco®) and Glutamax (Gibco®).

The medium obtained by mixing these components has a pH ranging from 5.5 to 9.0, preferably 6.0 to 8.0, more preferably 6.5 to 7.5.

The cell is cultured at 36° C. to 38° C., preferably 36.5° C. to 37.5° C., under conditions of 1% to 25% O₂ and 1% to 15% CO₂.

### 3.3 Measurement of Amount of TSLP Produced

In the present invention, the “amount of TSLP produced” used as an index is not limited to the physical quantity of TSLP and also encompasses activity or titer (antibody titer, etc.) indirectly representing this quantity. Likewise, the measurement of the amount of TSLP produced is not limited to measurement at the protein level and also encompasses measurement at the gene (mRNA) level.

Measurement of TSLP Production at Protein Level

The amount of SLP produced can be measured at the protein level using, for example, an immunological method that exploits antigen-antibody reaction. Examples of the immunological method can include: solid-phage immunoassay including immunoprecipitation, Western blotting, dot blotting, slot blotting, ELISA, and RIA; and modified versions thereof known in the art, such as sandwich ELISA, the method described in US Patent No. 4202875, and the method of Meager et al. (Meager A., Clin Exp Immunol. 2003 April, 132 (1), p. 128-36). Specifically, the amount of TSLP produced is measured using an anti-TSLP antibody specifically binding to TSLP on the basis of any of these methods.

The antibody used in the immunological method can be prepared according to a method known in the art or may be a commercially available product. The antibody can be obtained by immunizing an animal with antigenic TSLP or a portion thereof according to a routine method and collecting and purifying antibodies produced in vivo in the animal. Alternatively, antibody producing cells that produce specific antibodies may be fused with myeloma cells to establish hybridomas, from which monoclonal antibodies are in turn obtained according to a method known in the art (e.g., Kohler and Milstein, Nature 256, 495-497, 1975; and Kennett, R. ed., Monoclonal Antibodies p. 365-367, 1980, Plenum Press, N.Y.).

Examples of the antigen for antibody preparation used in detection can include antigenic TSLP or a portion (epitope region) thereof and derivatives thereof in which these antigens are conjugated with arbitrary carrier proteins (e.g., at the N termini with keyhole limpet hemocyanin).

The anti-TSLP antibody is directly labeled for detection. Alternatively, this antibody may be used as a primary antibody in detection in collaboration with a labeled secondary antibody that specifically recognizes the primary antibody (i.e., recognizes the antibody derived from the animal used in the antibody preparation).

The label is preferably an enzyme (alkaline phosphatase or horseradish peroxidase) or biotin (which requires an additional procedure of further binding enzyme-labeled streptavidin to the biotin in the secondary antibody), though its type is not limited to these labels. Various antibodies (or streptavidins) labeled in advance are commercially available as the labeled secondary antibody (or labeled streptavidin). In the case of RIA, an antibody labeled with a radioisotope such as ¹²⁵I is used and assayed using a liquid scintillation counter or the like.

The activity of such an enzyme used as a label is detected to measure the expression level of the antigen. For the alkaline phosphatase or horseradish peroxidase label, a substrate that develops color or emits light by the catalysis of this enzyme is commercially available.

In the case of using the substrate that develops color, this color can be detected by visual observation using Western blotting or dot/slot blotting. In ELISA, the absorbance (because a measurement wavelength differs depending on the type of the substrate) of each well is preferably measured using a commercially available microplate reader to quantify the antigen. Alternatively, dilution series of the antigen used in the antibody preparation may be prepared and used as standard antigen samples in detection operation together with other samples, and a standard curve plotting standard antigen concentrations and measured values is drawn to determine antigen concentrations in these other samples.

In the case of using the substrate that emits light, this light can be detected in Western blotting or dot/slot blotting by autoradiography using an X-ray film or an imaging plate or by photography using an instant camera. Alternatively, TSLP may be quantified using densitometry, Molecular Imager Fx System (manufactured by Bio-Rad Laboratories, Inc.), or the like. In the case of using the light-emitting substrate in ELISA, enzymatic activity is determined using a luminescence microplate reader (manufactured by, e.g., Bio-Rad Laboratories, Inc.).

Measurement of TSLP Production at Gene Level

The expression level of the TSLP gene is determined by first extracting total RNA from collected cells and measuring the expression level of the TSLP gene (mRNA) in this total RNA using any method described later.

The method for extracting total RNA is not particularly limited and can adopt, for example, ultracentrifugation using guanidine thiocyanate or cesium chloride, hot phenol method using guanidine thiocyanate, guanidine hydrochloride method, or acidic guanidine thiocyanate-phenol-chloroform method (Chomczynski, P. and Sacchi, N., (1987) Anal. Biochem., 162, 156-159). The extracted total RNA may be further purified, if necessary, into only mRNA.

The gene expression level can be measured using a method known in the art, such as nucleic acid hybridization using samples immobilized on solid phases such as gene chips or arrays, RT-PCR, real-time PCR, subtraction method, differential display method, differential hybridization, and cross-hybridization.

### 3.4 Evaluation of Test Substance

For evaluation, the amount of TSLP produced in the presence of a test substance may be compared with that in the absence of the test substance. Alternatively, provided that the reference value of standard TSLP production is determined, this reference value may be compared with the amount of TSLP produced in the presence of the test substance.

According to a standard embodiment, the method of the present invention comprises the steps of: 1) culturing the KCMH-1 cell line or variant thereof having a substantially equivalent TSLP productivity thereto in the presence and in the absence of a test substance; and 2) comparing the amount of TSLP produced between in the presence and in the absence of the test substance.

Specifically, if the amount of TSLP produced in the presence of a test substance is significantly different from the reference value or the amount of TSLP produced in the absence of the test substance, the test substance is selected as a TSLP modulator candidate. In this context, the term “significantly” means being statistical significant, for example, p<0.05, as usually used in the art.

In the case of screening for the TSLP production inhibitor such as an antiallergic agent or an anti-inflammatory agent, if the amount of TSLP produced in the presence of a test substance is significantly lower than the reference value or the amount of TSLP produced in the absence of the test substance, the test substance is selected as a TSLP production inhibitor candidate.

The screening method of the present invention employs the cell line that constantly exhibits high TSLP production without stimulation, and thus produces results with high accuracy and high reproducibility. Because of the exceedingly large amount of TSLP produced in KCMH-1, a high-throughput screening method can be constructed by scaling down the amount of the cells used. In addition, the screening method of the present invention can be used in the development of novel antiallergic agents intended to inhibit TSLP production.

The selected candidate substance targets the TSLP productivity of epithelial cells and as such, can be used as an external medicine or an inhalant. Also, the candidate substance targets TSLP and as such, can be used in the prevention of allergy or for a mild case of allergy with a low possibility of causing impaired immunity against infection. In addition, this compound has a low molecular weight and as such, can be used for a long period because of requiring lower cost than that of antibodies or soluble receptors.

### 4. Kit for Screening for TSLP Modulator

The present invention also provides a kit for screening for a TSLP modulator. The kit of the present invention comprises, as an essential component, a KCMH-1 cell line or variant thereof having a substantially equivalent TSLP productivity thereto.

The kit of the present invention may further comprise an anti-TSLP antibody or an anti-TSLP antibody and a secondary antibody capable of specifically binding to the anti-TSLP antibody.

The origin of the anti-TSLP antibody is not particularly limited as long as the antibody can be used in the detection of human TSLP. An anti-human TSLP antibody is preferable. The antibody may be labeled with an appropriate label (e.g., an enzymatic label, a radioactive label, or a fluorescent label) or may be modified appropriately with biotin or the like. The antibody may also be immobilized on an appropriate support. Alternatively, the kit may further comprise a support capable of immobilizing the antibody thereon. Examples of such a support that can be used include supports made of synthetic resins (e.g., polyethylene, polypropylene, polybutylene, polystyrene, polymethacrylate, and polyacrylamide) capable of being attached with proteins, glass, nitrocellulose, cellulose, or agarose, and gel-type supports. The support is provided in a form such as fine particles such as microspheres or beads (e.g., “latex” beads), a tube (inside wall) such as a microcentrifuge, or a microtiter plate (well), though the form is not particularly limited thereto.

The kit of the present invention may comprise, in addition to the components described above, other optional components necessary for carrying out the present invention, such as reagents for label detection, reaction buffer solutions, enzymes, and substrates.

### 5. Method for Producing Wild-Type TSLP

In the present invention, TSLP is wild-type TSLP produced by the KCMH-1 cell line or variant thereof described above. Currently commercially available TSLP is recombinant TSLP without sugar chains, which may differ in biochemical properties from TSLP produced by mammalian cells. The TSLP of the present invention is mouse TSLP having a sugar chain more analogous to that in human TSLP and can be obtained conveniently by culturing the KCMH-1 cell line or variant thereof according to the present invention.

The culture of the KCMH-1 cell line or variant thereof can be carried out according to the method described in the paragraph 3.1.

The present invention also provides a method for producing wild-type TSLP using the KCMH-1 cell line or variant thereof. The KCMH-1 cell line of the present invention constantly produces TSLP even without stimulation. The amount of TSLP produced by this cell line may be further enhanced by the addition of various cytokines. Thus, the cell may be cultured together with such a TSLP production stimulator added, without impairing the object of the present invention.

The KCMH-1 cell line or variant thereof secretes TSLP into its culture supernatant. The secreted TSLP can be collected from the culture supernatant according to a method known in the art. If TSLP does not have to be isolated, the culture supernatant may be subjected directly to subsequent procedures.

TSLP can be isolated using ion-exchange chromatography, gel filtration, reverse-phase HPLC, or the like according to the method of Sims et al. (Sims, J. E. et al. J. Exp. Med. (2000) 192: 671-680).

The TSLP or TSLP-containing culture supernatant thus prepared can also be used in the elucidation of TSLP-mediated pathogenesis of allergy or inflammation or the in-vitro evaluation of TSLP-targeting drugs.

In addition, the KCMH-1 cell line according to the present invention, and the screening method and the screening kit using this KCMH-1 cell line can also be exploited in analysis on the effects of signal transduction inhibitors or the elucidation of the mechanism underlying the induction of TSLP production, as shown in Examples described later.

### EXAMPLES

Hereinafter, the present invention will be described specifically with reference to Examples and Reference Examples. However, the present invention is not intended to be limited to these Examples.

### Reference Example 1

### Preparation of KCMH-1 Cell

KCMH-1 cells were established by Professor Hide (Faculty of Medicine, Hiroshima University) according to a method described below (see Arch Dermatol Res (1994) 287: 91-96).

0.2 ml of a solution of 3-methylcholanthrene in acetone (3 mg/ml) was applied to the dorsal skin of a CBA/J mouse (7 weeks old, male) twice a week for 3 weeks. Eight weeks later, 0.2 ml of a solution of 12-O-tetradecanoylphorbol-13-acetate in acetone (12 μg/ml) was applied to the same site. The formed skin cancer tissue was collected, and its slice of 1 mm square was transplanted into the thigh muscle of another CBA/J mouse. After 10 repetitive transplantations, the obtained cancer tissue was dispersed in an RPMI 1640 medium, and tissue pieces were filtered off through a 100-μm stainless sieve.

The resulting cells were cultured in an RPMI 1640 containing 10% FBS (containing 100 IU penicillin G and 100 μg/ml streptomycin). After 5 subcultures, the cells were inoculated at a concentration of 0.5 cells/well to a 96-well multiwell plate and cloned therein.

The established KCMH-1 cells were domestically deposited on May 21, 2010 under accession No. FERM P-21965 with International Patent Organism Depositary (IPOD), the National Institute of Advanced Industrial Science and Technology (AIST) (Tsukuba Central 6, 1-1-1 Higashi. Tsukuba, Ibaraki, Japan) and then transferred and internationally deposited therewith on Apr. 25, 2011 under accession No. FERM BP-11368.

### Example 1

### Comparison of Amount of TSLP Produced Among Keratinocyte-Derived Cells

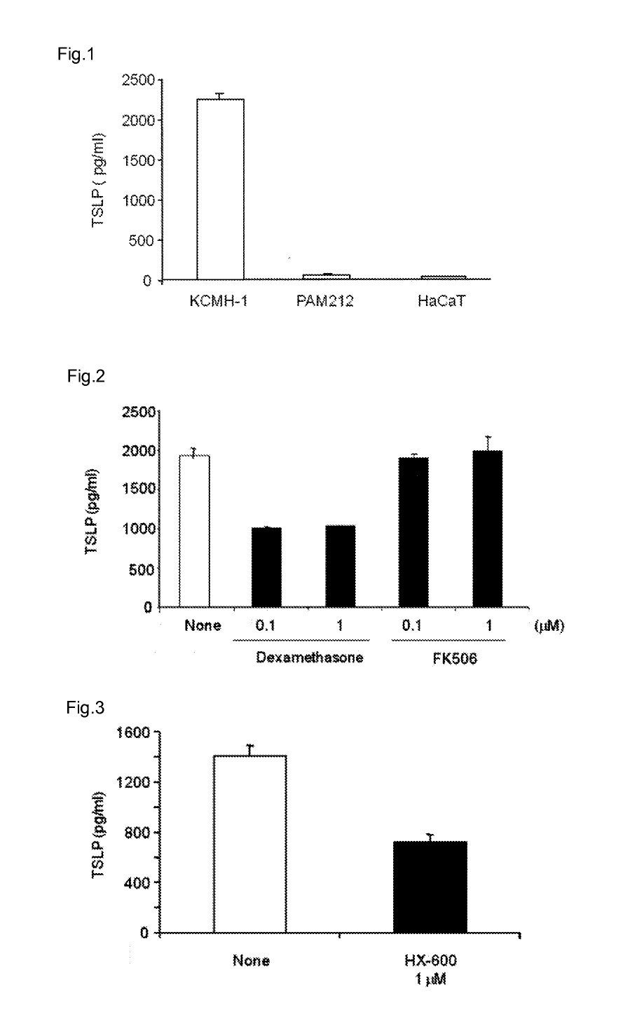

KCMH-1, mouse keratinocyte-like cell line PAM212 cells, and human keratinocyte-like cell line HaCaT cells were separately suspended at a concentration of 1×10⁵ cells/ml in an MEMU medium containing 10% FBS. 0.5 ml of each cell suspension was inoculated to each well of a 24-well cluster dish. 24 hours later, the culture solution was collected, and TSLP in the supernatant was quantified by ELISA (R&D Systems, Inc.) (FIG. 1).

As is evident from the results, the KCMH-1 cell line expresses TSLP at a significantly high level compared with other keratinocyte-derived cell lines.

### Example 2

### Inhibitory Effects of Dexamethasone and FK506 on TSLP Production of KCMH-1 Cell

The KCMH-1 cells were suspended at a concentration of 1×10⁵ cells/ml in an MEMU medium containing 10% FBS. 0.5 ml of the cell suspension was inoculated to each well of a 24-well cluster dish. 24 hours later, the medium was removed, and the cells were washed with phosphate-buffered saline (PBS). Then, 0.5 ml of an MEMU medium containing 10% FBS and each concentration of dexamethasone (0.1 or 1 μM) or FK506 (0.1 or 1 μ) was added to the cells. 24 hours later, the culture solution was collected, and TSLP in the supernatant was quantified by ELISA (FIG. 2).

As in the reports about normal human keratinocytes, it was confirmed that the amount of TSLP produced in the KCMH-1 cell line was evidently reduced by the addition of dexamethasone, but not by the addition of FK506.

The results described above are consistent with the results reported about normal keratinocytes (Le et al., Allergy 2009, 64, 1231-1232), demonstrating that the KCMH-1 cell line of the present invention is useful in cultured cell systems clearly suggesting inhibitory effects on TSLP production in response to known anti-inflammatory agents.

### Example 3

### Selective TSLP Productivity of KCMH-1 Cell

KCMH-1 and mouse keratinocyte-like cell line PAM212 cells were separately suspended at a concentration of 1×10⁵ cells/ml in an MEMα medium containing 10% FBS. 0.5 ml of each cell suspension was inoculated to each well of a 24-well cluster dish. 24 hours later, the culture solution was collected, and TSLP, IL-6, TNF-α, IL-4, and IFN-y in the supernatant were quantified by ELISA (R&D Systems, Inc. or eBioscience, Inc.). The results are shown in Table 1.

TABLE 1

| | Production (pg/ml) | |
|---|---|---|
| | KCMH-1 | PAM212 |
| TSLP | 2671 ± 266 | 7 ± 5 |
| IL-6 | 18 ± 1 | 15 ± 2 |
| TNF-α | 16 ± 2 | N.D. |
| IL-4 | 5 ± 1 | N.D. |
| IFN-γ | 4 ± 2 | N.D. |

N.D.: notdetermined

As is evident from the results, it was confirmed that the KCMH-1 cell line highly produced TSLP in a selective manner compared with other cytokines and that this feature was not observed in other keratinocyte-derived cell lines.

### Example 4

### Inhibitory Effect of RXR Agonist on TSLP Production of KCMH-1 Cell

HX-600, an agonist of retinoid X receptor (RXR), was allowed to act on KCMH-1 and examined for its effect on the TSLP production thereof.

The KCMH-1 cells were suspended at a concentration of 1×10⁵ cells/ml in an MEMU medium containing 10% FBS. 0.5 ml of the cell suspension was inoculated to each well of a 24-well cluster dish. 24 hours later, the medium was removed, and the cells were washed with phosphate-buffered saline (PBS). Then, 0.5 ml of an MEMU medium containing 10% FBS and each concentration of HX-600 (1 μM) was added to the cells. 24 hours later, the culture solution was collected, and TSLP in the supernatant was quantified by ELISA (FIG. 3).

The reported experiment on nuclear receptor stimulators using human airway epithelial cell lines have revealed that the RXR agonist 9-cis-retinoic acid (9-cis-RA) inhibits the expression of human TSLP mRNA by IL-1β (Lee et al. J. Immunol. 181: 5189-5193 (2008)). The results described above are consistent with the report about human airway epithelial cell lines, demonstrating that the RXR agonist also has inhibitory activity on the TSLP production of the KCMH-1 cell line.

### Example 5

### Effect of Signal Transduction Inhibitor on KCMH-1 Cell

Various tyrosine kinase inhibitors were allowed to act on the KCMH-1 cells, and their influence on TSLP production was compared. The tyrosine kinase inhibitors used were herbimycin A, PP2, piceatannol, AG490, and WHI-P154. Herbimycin is known as a nonspecific tyrosine kinase inhibitor, whereas PP2, piceatannol, AG490, and WHI-P154 are known to inhibit Src family, Syk, JAK2, and JAK3, respectively.

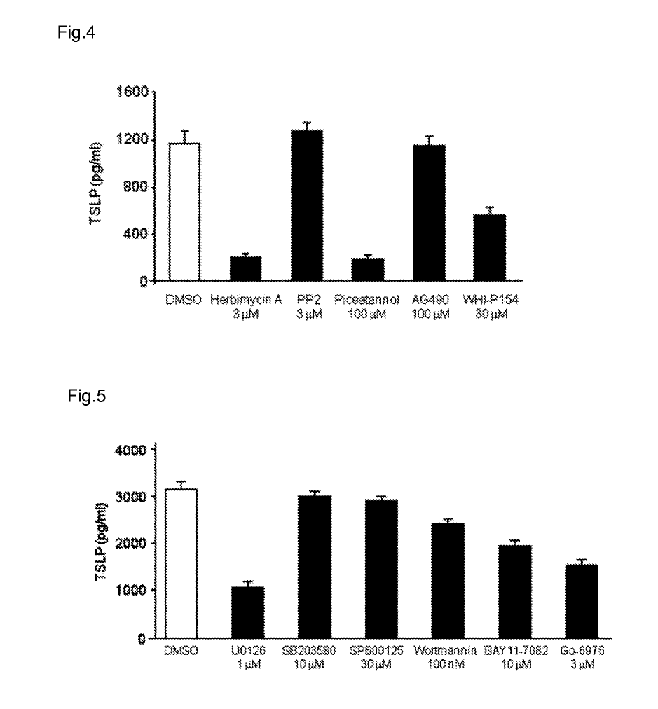

The KCMH-1 cells were suspended at a concentration of 1×10⁵ cells/ml in an MEMU medium containing 10% FBS. 0.5 ml of the cell suspension was inoculated to each well of a 24-well cluster dish. 24 hours later, the medium was removed, and the cells were washed with phosphate-buffered saline (PBS). Then, 0.5 ml of an MEMU medium containing 10% FBS and herbimycin A (3 μM), PP2 (3 μM), piceatannol (100 μM), AG490 (100 μM), or WHI-P154 (30 μM) at the final concentration shown within the parentheses was added to the cells. 24 hours later, the culture solution was collected, and TSLP in the supernatant was quantified by ELISA (FIG. 4).

Subsequently, various serine-threonine kinase inhibitors were allowed to act on the KCMH-1 cells, and their influence on TSLP production was compared. The serine-threonine kinase inhibitors used were U0126, SB203580, SP600125, wortmannin, BAY11-7082, and Go-6976. In general, U0126 is an activation inhibitor for p44/42 MAP kinase, whereas Go-6976 is a protein kinase C inhibitor known to inhibit the production or release of various proteins. By contrast, wortmannin is known as a PI3 kinase inhibitor; SB203580 is known as a p38 MAP kinase inhibitor; and SP600125 is known as a c-Jun N-terminal kinase inhibitor. BAY11-7082 is known to inhibit IKB kinase to inhibit the activation of NF-κB.

The KCMH-1 cells were suspended at a concentration of 1×10⁵ cells/ml in an MEMU medium containing 10% FBS. 0.5 ml of the cell suspension was inoculated to each well of a 24-well cluster dish. 24 hours later, the medium was removed, and the cells were washed with phosphate-buffered saline (PBS). Then, 0.5 ml of an MEMU medium containing 10% FBS and U0126 (1 μM), SB203580 (10 μM), SP600125 (30 μM), wortmannin (100 nM), BAY11-7082 (10 μM), or Go-6976 (3 μM) at the final concentration shown within the parentheses was added to the cells. 24 hours later, the culture solution was collected, and TSLP in the supernatant was quantified by ELISA (FIG. 5).

According to the previous report, the transcriptional factor NF-KB is involved in intracellular signal transduction associated with TSLP production in human airway epithelial cell lines (Lee and Ziegler, Proc. Natl. Acad. Sci. USA 104: 914-919 (2007)). It was confirmed that The NF-KB inhibitor BAY11-7082 also partially inhibited TSLP production in the KCMH-1 cells. In addition, unidentified tyrosine kinase and p44/42 MAP kinase were newly shown to contribute to TSLP production. Thus, use of the KCMH-1 cells allows analysis on the mechanism of signal transduction associated with TSLP production.

All publications, patents, and patent applications cited herein are incorporated herein by reference in their entirety.

### INDUSTRIAL APPLICABILTY

According to the present invention, the TSLP-modulating activity of a test substance can be evaluated conveniently in vitro. A substance having TSLP-modulating activity may be used as an antiallergic agent or an anti-inflammatory agent. Thus, the present invention can be used in a convenient and inexpensive screening system for such drugs. Particularly, the KCMH-1 cell line used in the present invention is derived from a keratinocyte and as such, is useful in screening for therapeutic or preventive drugs for cutaneous allergy or searching for the mechanism underlying this allergy.

According to the present invention, wild-type TSLP can be obtained conveniently. The obtained TSLP can also be used in the elucidation of TSLP-mediated pathogenesis of allergy and inflammation or screening for TSLP modulators.

1. A method for screening for a TSLP modulator, comprising: allowing a test substance to act on a KCMH-1 cell line specified by accession No. FERM BP-11368 or variant thereof having a substantially equivalent TSLP productivity thereto; and measuring the obtained amount of TSLP produced.

2. The method according to claim 1, comprising the following steps:
1) culturing the KCMH-1 cell line or variant thereof having a substantially equivalent TSLP productivity thereto in the presence and in the absence of the test substance; and
2) comparing the amount of TSLP produced between in the presence and in the absence of the test substance.

3. The method according to claim 1, wherein the amount of TSLP produced is measured using an anti-TSLP antibody specifically binding to TSLP.

4. The method according to claim 3, wherein the amount of TSLP produced is measured by Western blotting, dot blotting, slot blotting, ELISA, RIA, or flow bead array assay.

5. The method according to claim 1, wherein the TSLP modulator is a TSLP production inhibitor.

6. The method according to claim 5, wherein if the amount of TSLP produced in the presence of a test substance is significantly lower than that in the absence of the test substance, the test substance is selected as a TSLP production inhibitor candidate.

7. The method according to claim 5, wherein the TSLP production inhibitor is an antiallergic agent or an anti-inflammatory agent.

8. A kit for screening for a TSLP modulator, comprising a KCMH-1 cell line or variant thereof having a substantially equivalent TSLP productivity thereto.

9. The kit according to claim 8, further comprising an anti-TSLP antibody or an anti-TSLP antibody and a secondary antibody capable of specifically binding to the anti-TSLP antibody.

10. A method for producing TSLP, comprising culturing a KCMH-1 cell line specified by accession No. FERM BP-11368 or variant thereof having a substantially equivalent TSLP productivity thereto.
